(19) United States
(12) Patent Application Publication
(10) Pub. No.: US 2026/0335380 A1
(43) Pub. Date: Oct. 29, 2026

(54) MICROBIOME COMPOSITION OF HALOPHILIC BACILLUS VELEZENSIS KMU01 STRAIN FERMENTATION CULTURE SUPERNATANT WITH ANTI-OBESITY EFFICACY

(71) Applicant: KOOKMINBIO, CORP., Jeonju-si, Jeollabuk-do (KR)

(72) Inventor: Moon-Hee SUNG, Seoul (KR)

(73) Assignee: KOOKMINBIO, CORP., Jeonju-si, Jeollabuk-do (KR)

(21) Appl. No.: 19/122,259

(22) PCT Filed: Oct. 18, 2023
(86) PCT No.: PCT/KR2023/016092

§ 371 Date: Apr. 18, 2025

(30) Foreign Application Priority Data
Oct. 18, 2022 (KR) 10-2022-0134157
Oct. 12, 2023 (KR) 10-2023-0135572

(51) Int. Cl. A61K 35/742; A61K 9/00; A61K 31/19; A61K 31/198; A61P 3/04

(52) U.S. Cl. CPC A61K 35/742; A61K 9/0053; A61K 31/19; A61K 31/198; A61P 3/04

(57) ABSTRACT

The present invention relates to a microbiome composition of halophilic Bacillus polyfermenticus KMU01 strain fermentation culture supernatant with anti-obesity efficacy, wherein the fermentation culture supernatant of the strain deposited with accession number KCTC11751BP has been confirmed to inhibit the generation and accumulation of fat and to reduce the content of blood cholesterol, and thus is useful as a composition for preventing, treating, or ameliorating obesity or as a composition for reducing body fat (visceral fat) or blood cholesterol.

### TECHNICAL FIELD

The present disclosure relates to a microbiome composition of a fermentation culture supernatant of a halophilic Bacillus velezensis KMU01 strain with an anti-obesity efficacy.

### BACKGROUND ART

Obesity is the most representative disease among those with disease patterns that are rapidly changing to those of advanced countries due to the improvement of living standards that led to improved sanitary environments as well as westernized diets that extended average life expectancy. Accordingly, adult diseases have emerged as the biggest medical challenge today, and obesity, which is the main cause of these adult diseases, is also rapidly increasing.

Obesity, which is a disease caused by an imbalance between food intake and energy consumption, refers to a condition in which adipose tissues increase excessively. Persistent obesity causes various diseases such as high blood pressure, elevated blood cholesterol, kidney diseases, stroke, arteriosclerosis, fatty liver, arthritis, cancer, sleep apnea, and diabetes, among which the accumulation of visceral fat in the abdominal area causes insulin resistance or increased lipogenesis in the liver to lead to abnormal glucose and lipid metabolism, high blood pressure, and coronary artery diseases, such that the importance of obesity treatment is being emphasized.

Treatments for obesity are generally divided into three categories including appetite suppressants, stimulants for body energy metabolism, and digestion and absorption inhibitors. A representative obesity treatment that uses a pharmacological mechanism to suppress appetite includes Reductil™ (Abbott, USA), a representative obesity treatment that uses a pharmacological mechanism to boost body energy includes Exorise™ (Aco Pharma, France), and a representative obesity treatment that uses a pharmacological mechanism to suppress fat digestion and absorption includes Xenical™ (Roche, Switzerland).

Recently, with the advancement of next-generation sequencing (NGS) technology, many studies on microorganisms existing in the human body are being conducted, and research to prove the correlation between obesity and the gut microbiome is also actively underway. The gut microbiome is closely related to the host's diet, and it has been reported that obesity can lead to dysbiosis of intestinal microbes by causing changes in the colonies and functions of intestinal microbes. In fact, when the intestinal microbes from obese mice were transplanted into normal or germ-free mice, weight gain and metabolic disorders took place, with a report that the intestinal microbes are related to the regulation of dietary energy utilization and fatty acid metabolism in adipose and liver tissues. Therefore, modulation of the intestinal microbial colonies may be a non-toxic, safe, and potential treatment for metabolic disorders in the amelioration of obesity.

Although probiotics have the effect of preventing and treating immune diseases by controlling the intestinal flora and blocking the access of pathogenic microorganisms, but they are unstable and has a poor ability to reach the intestines, and thus a cell-free supernatant (CFS) is attracting attention as a new alternative material that may overcome safety and functionality issues due to side effects caused by excessive intake. Recently, culture media of Bifidobacterium bifidum DS0908 and Bifidobacterium bifidum DS0905 have been shown to reduce obesity by promoting thermogenesis in obese mice, and short-chain fatty acids (SCFAs) have been reported to prevent obesity induced by a high-fat diet by regulating intestinal hormones. As such, various studies on the physiological activity of strain culture media are in progress.

### DISCLOSURE OF THE INVENTION

### Technical Goals

An object of the present disclosure is to provide a pharmaceutical composition for preventing or treating obesity.

Another object of the present disclosure is to provide a health functional food composition for preventing or ameliorating obesity.

Another object of the present disclosure is to provide a food composition for preventing or ameliorating obesity.

Another object of the present disclosure is to provide a health functional food composition for reducing body fat or blood cholesterol.

Another object of the present disclosure is to provide a method of preventing or treating obesity.

### Technical Solutions

To achieve the above objects, the present disclosure provides a pharmaceutical composition for preventing or treating obesity, including a fermentation culture supernatant of a Bacillus velezensis strain, a concentrate thereof, a dried product thereof, a fermentation metabolite thereof, or a mixture thereof as an active ingredient.

In addition, the present disclosure provides a health functional food composition for preventing or ameliorating obesity, including a fermentation culture supernatant of the strain, a concentrate thereof, a dried product thereof, a fermentation metabolite thereof, or a mixture thereof as an active ingredient.

In addition, the present disclosure provides a food composition for preventing or ameliorating obesity, including a fermentation culture supernatant of the strain, a concentrate thereof, a dried product thereof, a fermentation metabolite thereof, or a mixture thereof as an active ingredient.

In addition, the present disclosure provides a health functional food composition for reducing body fat or blood cholesterol, including a fermentation culture supernatant of the strain, a concentrate thereof, a dried product thereof, a fermentation metabolite thereof, or a mixture thereof as an active ingredient.

In addition, the present disclosure provides a method of preventing or treating obesity, including treating a subject with the pharmaceutical composition for preventing or treating obesity.

### Advantageous Effects

According to the present disclosure, it was found that a fermentation culture supernatant of a Bacillus velezensis KMU01 strain deposited under deposit number KCTC11751BP inhibits fat production and accumulation and reduces a blood cholesterol content, such that it may be usefully utilized as a composition for preventing, treating, or ameliorating obesity; or a composition for reducing body fat (visceral fat) or blood cholesterol.

### BRIEF DESCRIPTION OF DRAWINGS

FIG. 1 is a schematic diagram showing an adipogenesis process using 3T3-L1 preadipocytes.

FIG. 2 is an image obtained by photographing a process of adipose tissue extraction in a mouse animal model.

FIG. 3 is a schematic diagram showing an intestinal microbe analysis process of a mouse animal model.

FIG. 4 shows a result of analyzing a cytotoxicity of a culture solution (hereinafter referred to as a sample) of a Bacillus velezensis KMU01 (hereinafter referred to as KMU01) strain in adipocytes.

FIG. 5 shows results of analyzing an effect of a sample on accumulation of fat and triglyceride (hereinafter referred to as TG).

FIG. 6 shows results of analyzing an effect of a sample on expression of genes related to adipogenesis and enzymes related to lipogenesis (fatty acid synthase; hereinafter referred to as FAS).

FIG. 7 shows results of analyzing an effect of a sample on a body weight and food efficiency of an animal model.

FIG. 8 shows results of analyzing an effect of a sample on a body composition of an animal model.

FIG. 9 shows results of analyzing an effect of a sample on organs and adipose tissues of an animal model. iWAT; inguinal white adipose tissue, mWAT; mesenteric white adipose, rWAT; retroperitoneal white adipose tissue and eWAT; epididymal white adipose tissue.

FIG. 10 shows results of analyzing an effect of a sample on adipose tissues of an animal model.

FIG. 11 shows a result of analyzing an effect of a sample on hepatic TG of an animal model.

FIG. 12 shows results of analyzing an effect of a sample on expression of proteins related to adipogenesis and lipogenesis in liver of an animal model.

FIG. 13 shows results of analyzing an effect of a sample on intestinal microbes of an animal model.

It is noted that all drawings have statistical differences among treated groups represented by each different letter (e.g. a, b, etc.) at a 95% confidence level. For example, if there are group 1 represented as a, group 2 as b, and group 3 as ab, it is indicated that there is a statistical difference at a 95% level between groups 1 and 3, and there is no statistical difference between groups 1 and 2; and between groups 2 and 3.

### BEST MODE FOR CARRYING OUT THE INVENTION

Hereinafter, the present disclosure will be described in more detail.

The present disclosure provides a pharmaceutical composition for preventing or treating obesity, including a fermentation culture supernatant of a Bacillus velezensis strain, a concentrate thereof, a dried product thereof, a fermentation metabolite thereof, or a mixture thereof as an active ingredient.

The strain may be a Bacillus velezensis KMU01 strain deposited under deposit number KCTC11751BP.

The strain name of the Bacillus velezensis KMU01 strain at the time of deposit is Bacillus KMU01. Specifically, the KMU01 strain was isolated as Bacillus amyloliquefaciens in 2010 and reclassified as Bacillus polyfermenticus in 2018 based on the 16S rRNA gene sequence. Afterwards, an experiment conducted to accurately identify the species of the KMU01 strain had revealed that the genetic sequence of the KMU01 strain showed 97.7% similarity to Bacillus velezensis, and the KMU01 strain was currently determined as Bacillus velezensis (Functional Annotation Genome Unravels Potential Probiotic Bacillus velezensis Strain KMU01 from Traditional Korean Fermented Kimchi, DOI: https://doi.org/10.3390/foods10030563, released on 2021.05.09.).

Recently, the Bacillus polyfermenticus KMU01 strain was renamed Bacillus velezensis (Genome Sequence of the Probiotic Strain Bacillus velezensis Variant polyfermenticus GF423, DOI: 10.1128/MRA.01000-18, released on 2018.09.13.).

The pharmaceutical composition may further include killed cells or spores of Bacillus velezensis.

The fermentation metabolite may be short chain fatty acids (SCFAs), organic acids, or amino acids.

The short-chain fatty acid may be butyric acid or propionic acid, and the amino acid may be phenylalanine, which is an aromatic amino acid, or valine, which is a branched amino acid, but are not limited thereto.

In addition, the pharmaceutical composition may regulate adiponectin secretion.

In addition, the pharmaceutical composition may regulate one or more intestinal microbes selected from the group consisting of, but not limited to, Acetatifactor muris, Mucispirillum schaedleri, and Eubacterium plexicaudatum.

In addition, the pharmaceutical composition may inhibit expression of one or more selected from the group consisting of peroxisome proliferator-activated receptor γ (PPARγ), CCAAT/enhancer binding protein α (C/EBPα), sterol regulatory element-binding protein-1c (SREBP-1c), fatty acid synthase (FAS), acetyl-CoA carboxylase (ACC), stearoyl-CoA desaturase-1 (SCD-1), and diacylglycerol acyltransferase (DGAT), but is not limited thereto.

The obesity may be one or more selected from the group consisting of visceral obesity, abdominal obesity, systemic obesity, and partial obesity, but is not limited thereto.

The pharmaceutical composition of the present disclosure may be prepared in the form of a unit dose by formulation using a pharmaceutically acceptable carrier or prepared by encapsulating into a multi-capacity container, in accordance with a method that may be easily carried out by a person with ordinary skill in the art to which the disclosure pertains.

The pharmaceutically acceptable carriers are commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additives included in the pharmaceutical composition is not particularly limited and may be appropriately adjusted within a content range used in conventional preparation.

The pharmaceutical composition may be formulated in the form of one or more external skin agents selected from the group consisting of, but is not limited to, injectable formulations such as aqueous solutions, suspensions, and emulsions, pills, capsules, granules, tablets, creams, gels, patches, sprays, ointments, plasters, lotions, liniments, pastas, and cataplasmas.

The pharmaceutical composition of the present disclosure may include a pharmaceutically acceptable carrier and diluent that are additionally present for formulation. The pharmaceutically acceptable carriers and diluents include, but are not limited to, excipients such as starch, sugars, and mannitol, fillers and bulking agents such as calcium phosphate, cellulose derivatives such as carboxymethylcellulose and hydroxypropylcellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, and elixirs. When administered parenterally, it may be formulated as injection solutions, suppositories, powders for respiratory inhalation, aerosols for sprays, ointments, powders for application, oils, and creams.

The dosage range of the pharmaceutical composition of the present disclosure may vary depending on the patient's condition, weight, age, sex, health status, dietary constitution specificity, nature of the preparation, severity of a disease, administration time for composition, method of administration, duration or interval of administration, excretion rate, and drug form, and may be appropriately selected by a person skilled in the art. For example, it may range from about 0.1 to 10,000 mg/kg, but is not limited thereby, and it may be administered once or several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, type of administration, administration time, and administration route, and a person with ordinary skill in the art may easily determine and prescribe the effective dosage for the desired treatment. The administration of the pharmaceutical composition of the present disclosure may be administered once a day or in several divided doses.

In addition, the present disclosure provides a health functional food composition for preventing or ameliorating obesity, including a fermentation culture supernatant of a Bacillus velezensis strain, a concentrate thereof, a dried product thereof, a fermentation metabolite thereof, or a mixture thereof as an active ingredient.

In addition, the present disclosure provides a health functional food composition for reducing body fat or blood cholesterol, including a fermentation culture supernatant of a Bacillus velezensis strain, a concentrate thereof, a dried product thereof, a fermentation metabolite thereof, or a mixture thereof as an active ingredient.

The strain may be a Bacillus velezensis KMU01 strain deposited under deposit number KCTC11751BP.

The present disclosure may be used generally as a food product that is conventionally used.

The food composition of the present disclosure may be used as a health functional food. The term “health functional food” as used herein refers to a food manufactured and processed using raw materials or ingredients that have useful functionality for the human body in accordance with the Health Functional Food Act, and the term “functionality” as used herein refers to consumption for the purpose of deriving useful effects for health purposes such as adjusting nutrients or physiological actions on the structure and function of the human body.

The health functional food composition may include conventional food additives, and the suitability as the “food additive” is determined by the standards and criteria associated with corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying obesity by administering the composition according to the present disclosure.

The term “treatment” as used herein refers to any action that ameliorates or favorably changes symptoms of obesity by administering the composition according to the present disclosure.

The term “amelioration” as used herein refers to any action of making the bad condition of obesity better by administering or ingesting the composition according to the present disclosure to a subject.

In addition, the present disclosure provides a functional food including the health functional food composition.

In addition, the present disclosure provides a food composition for preventing or ameliorating obesity, including a fermentation culture supernatant of a Bacillus velezensis strain, a concentrate thereof, a dried product thereof, a fermentation metabolite thereof, or a mixture thereof as an active ingredient.

The strain may be a Bacillus velezensis KMU01 strain deposited under deposit number KCTC11751BP.

In addition, the present disclosure provides a method of preventing or treating obesity, including treating a subject with the pharmaceutical composition for preventing or treating obesity.

The method of preventing or treating obesity may derive an effect of ameliorating obesity or reducing visceral fat by decreasing a relative abundance of Acetatifactor muris or Mucispirillum schaedleri strains in the intestine and increasing a relative abundance of a Eubacterium plexicaudatum strain in the intestine.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Experimental Example 1] Sample Preparation

To prepare a sample, the KMUO1 (Bacillus velezensis KMU01) strain deposited under stock deposit number KCTC11751BP stored in the −70 °C. Working cell bank was activated and subjected to primary seed culture in test tubes and flasks, and then 2% (v/v) thereof was inoculated in 20 L working volume in a 50 L fermenter, followed by secondary seed culture for 6 hours. This culture was performed by inoculating 2% (v/v) in 350 L working volume in a 500 L fermenter for 12 hours, and glucose was additionally fed once at 6 hours of culture. After completion of the culture, the cell slurry was removed by performing the primary centrifugation at 7,200 rpm and 2 L/min in a disk centrifuge, and the supernatant was secondarily centrifuged twice at 15,000 rpm and 1.5 L/min in a tubular centrifuge to remove the cell cake and collect the supernatant. The recovered supernatant was filtered through a 0.2 μm sterilizing filter to obtain a sample from which the bacterial cells were finally removed.

### [Experimental Example 2] In Vitro Experiment

### 2-1. Cell Culture and Differentiation

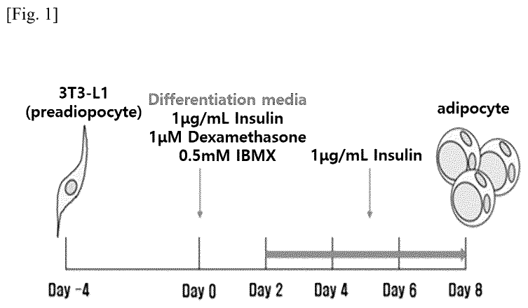

3T3-L1 (ATCC, Manassas, VA, USA) fibroblasts, which are preadipocytes, were cultured in Dulbecco's modified Eagle's medium (DMEM) containing 10% (v/v) bovine calf serum and 100 μg/mL penicillin-streptomycin at 37 °C in the presence of 5% CO₂. Thereafter, as shown in FIG. 1, when the cells reached 100% confluency, differentiation into adipocytes was induced in DMEM containing 10% fetal bovine serum (FBS), 1 μM dexamethasone, 0.5 mM isobutyl methylxanthine (IBMX), 1 μg/mL insulin, and 100 μg/mL penicillin-streptomycin. Two days after differentiation, the medium was replaced with 10% FBS containing 1 μg/mL insulin and the sample (the medium was replaced every other day), and the effect on the proliferation and differentiation of adipocytes was analyzed on the 8th day.

### 2-2. Cytotoxicity Assay

To identify the cytotoxicity of the sample in adipocytes, an MTT assay was performed. Samples were treated by concentrations (75, 150, and 300 μg/mL) to adipocytes (3T3-L1), and the cell viability was measured by conducting an MTT assay.

### 2-3. Analysis of Adipose Accumulation and TG Content

To identify an effect of the sample on fat accumulation, the sample was treated to adipocytes (3T3-L1) by concentrations (75, 150, and 300 μg/mL), and the fat accumulation rate of mature adipocytes was measured by staining the adipocytes with Oil-Red O (ORO) staining.

In addition, to identify the effect of the sample on TG, the content of TG accumulated in the cells was measured using a TG quantification kit (Abcam, Cambridge, MA, USA), the protein content was quantified via bicinchoninic acid (BCA) analysis, and then the TG content of the cells was expressed by correcting by protein concentration.

### 2-4. Analysis of Adipogenesis-Related Genes and FAS Expression

To identify an effect of the sample on adipogenesis-related genes and FAS expression, RNA was extracted using Nucleozol (Macherey-Nagel, Düren, Germany) reagent, and cDNA was synthesized using a reverse transcription kit (Applied Biosystem, Foster city, CA, USA). Afterwards, the expression of adipogenesis-related genes (PPARy, C/EBPα, and SREB-1c) and FAS was analyzed using the StepOnePlus Real-Time PCR (Quantitative real-time PCR; qPCR) system (Applied Biosystem), and the gene expression level was corrected using the glycealdehyde-3-phosphate dehydrogenase (GAPDH) gene.

### 2-5. Statistical Analysis

All quantitative analyses were performed three times. Statistical analysis was performed using SPSS (SPSS Inc., USA) software, and when a significant difference was observed in one-way analysis of variance (ANOVA) (P<0.05), Duncan's multiple comparison method was conducted to test significant differences between treated groups.

### [Experimental Example 3] In Vivo Experiment

### 3-1. Preparation of Animal Models

C57BL/6J 5-week-old male mice were purchased from RAONBIO Inc. (Republic of Korea). Upon entry, the animals were inspected for appearance and their weights were measured. During the acclimatization period, general symptoms were observed once a day, and at the end of the acclimatization period, body weight was measured, and general symptoms and weight changes were checked to evaluate the health status of the animals. To ensure evenness in the average weight of each experimental group, the animals were divided into six groups in total, each with about 10 animals, and housed with five animals per cage. Individual marks were made on the animals' tails using a five-colored permanent marker, and individual identification cards were attached to their breeding boxes. Animal models were raised for 2 weeks under conditions with a temperature of 21~23 °C, relative humidity of 40~60%, and a 12-h/day light/dark cycle (8:00 AM~8:00 PM), with a supply of food and drinking water. The feed used was experimental animal feed (6% fat feed and 45% fat feed) (ENVIGO, RESEARCH DIETS Inc.). Animal experiments were conducted in accordance with the Standard Operating Procedures of Kookmin University with the approval by the Institutional Animal Care and Use Committee at Kookmin University (KMU-2022-01).

### 3-2. Analysis of the Body Weight and Food Efficiency

In order to identify the effect of the sample on the body weight and food efficiency of the animal model, the animal model (7 weeks old) prepared in the Experimental Example 3-1 was set into four groups as follows, the body weight and feed intake were measured at weekly intervals for 13 weeks, and the food efficiency was calculated using the following Equation 1. Xenical was used as a positive control. Samples and Xenical were administered orally into the stomach once daily for 13 weeks from the start of administration using a disposable syringe with an oral administration sonde attached.
1) A normal diet group (NOR): 6% fat feed intake group
2) A high-fat diet group (HFD): 45% fat feed intake group
3) A sample administered group (B. vele): a high-fat diet group (HFD) orally administered with a sample (114 mg/kg/day)
4) A positive control group (Xen): a high-fat diet group (HFD) administered with Xenical (50 mg/kg/day)

Food efficiency ratio=Weight gain (g/week)/Food intake (g/week)×100 [Equation 1]

### 3-3. Analysis of a Body Composition

To identify an effect of the sample on the body composition of the animal model, changes in the body composition of the animal model were measured 13 weeks before sacrifice using dual energy X-ray absorptiometry (InAlyzer; Medikors Inc., Seongnam, Korea). When analyzing the body composition, animal models were anesthetized by injection of ketamine (100 mg/kg BW) and xylazine (10 mg/kg BW), and then measurements were followed. When only ketamine, which has an anesthetic effect, is used, side effects due to muscle contraction may occur during the recovery from the anesthesia, and thus xylazine, a muscle relaxant, was used together.

### 3-4. Analysis of Weights of Organs and Adipose Tissues

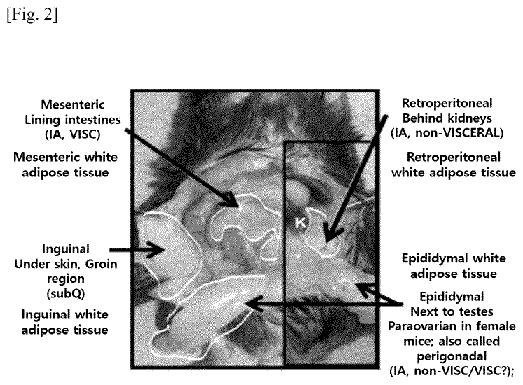

To identify an effect of the sample on organs and adipose tissues in the animal model, the animal models were fasted for 18 hours, sacrificed, and dissected, and then the heart, liver, kidneys, and spleen were removed to measure the weights of the organs. As shown in FIG. 2, the adipose tissue was separated into inguinal white adipose tissue, mesenteric white adipose tissue, retroperitoneal white adipose tissue, and epididymal white adipose tissue to measure the weight.

### 3-5. Blood Biochemical Analysis

To identify an effect of the sample on blood glucose, aspartate aminotransferase (GOT; hereinafter referred to as AST), alanine aminotransferase (GPT; hereinafter referred to as ALT), blood urea nitrogen (hereinafter referred to as BUN), and cholesterol in animal models, blood was collected from the heart after sacrificing the animal models and then immediately centrifuged (2000×g, 10 minutes) to separate plasma, and plasma was stored in a −80 °C deep freezer until analysis. Blood glucose, AST, ALT, and BUN were measured using a chemical analyzer (Fuji DRI-CHEM 3500i, Fuji Photo Film, Ltd., Tokyo, Japan), the total cholesterol and high density lipoprotein (HDL)-cholesterol (hereinafter referred to as HDL-C) were measured using a LabAssay™ Cholesterol kit (Wako, Osaka, Japan), and low density lipoprotein (LDL)-cholesterol (hereinafter referred to as LDL-C) was calculated using the following Equation 2. In addition, TG was analyzed using each TG assay kit (Abcam, Cambridge, MA).

LDL-C=Total cholesterol-{(HDL-C)+(TG/5)} [Equation 2]

### 3-6. Histological Analysis

To identify an effect of the sample on adipose tissues in the animal model, epididymal white adipose tissue (eWAT) and liver sections of the animal model were fixed in 10% formaldehyde, and paraffin blocks were created, which were then subjected to staining with H&E (Hematoxylin & Eosin). The size of the adipocytes was expressed as the average after obtaining the areas of 15 adipocytes in the center of representative images using KFBIO Slide Manager (KFBIO, Ningbo, China).

### 3-7. Analysis of a TG Content in the Liver

To identify an effect of the sample on TG in the liver of the animal model, the liver tissue of the animal model was crushed, and then TG was extracted for analysis using a TG assay kit (Abcam).

### 3-8. Analysis of Protein Expression Related to Hepatic Adipogenesis and Lipogenesis

To identify an effect of the samples on the expression of proteins related to hepatic adipogenesis and lipogenesis in the animal model, hepatic tissues were homogenized using a bullet blender (Next Advance, Troy, NY, USA) in radioimmunoprecipitation assay (RIPA) buffer containing 1% protease inhibitor and 1% phosphatase inhibitor to be used in the experiment. The homogenized tissue was left at 4 °C for 50 minutes and centrifuged at 4 °C and 15,000×g for 15 minutes to obtain the supernatant. Equal amounts of proteins were separated on 10% SDS-PAGE and transferred to polyvinylidene fluoride membranes (Bio-Rad, Hercules, CA, USA). After blocking with a blocking buffer solution with 5% bovine serum albumin-containing Tris-buffered saline solution with Tween 20 (hereinafter referred to as TBST, 0.1%), reaction was carried out overnight at 4 °C with ACC, p-ACC, FAS, C/EBPα, PPARγ, SCD-1, SREBP-1c, DGAT, and β-actin antibodies. After reacting with horseradish peroxidase-labeled secondary antibody at room temperature for 1 hour, washing was followed four times with TBST buffer. Protein bands were detected using enhanced chemiluminescence detection kits (BioRad, Hercules, CA, USA), and band intensities were quantified using Image Lab software 5.1 (BioRad) after correction for β-actin protein.

### 3-9. Analysis of Intestinal Microbes

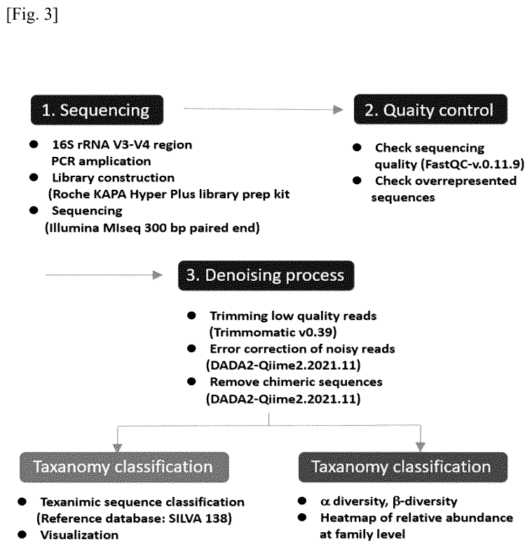

To identify an effect of the sample on the intestinal microbes of the animal model, the animal model was sacrificed, the cecum was removed, and analysis was performed using 16s rRNA metagenome sequencing, as shown in FIG. 3.

### 3-10. Statistical Analysis

GraphPad Prism 9.4.0 (GraphPad Software Inc., San Diego, CA, USA) and SPSS statistics V. 26 (SPSS Inc., Chicago, IL, USA) were used for statistical analysis, and significant differences (p<0.05) between treated groups were analyzed using one-way analysis of variance (ANOVA) and Duncan and Newman-Keuls' multiple comparison test.

### [Example 1] In Vitro Experiment

### 1-1. Cytotoxicity Assay

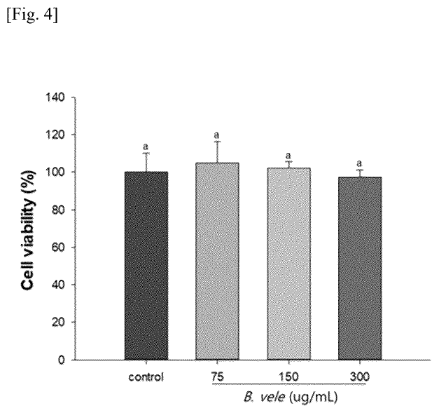

According to the Experimental Example 2-2, as a result of analyzing a cytotoxicity of the sample in adipocytes, as shown in FIG. 4, it was found that there was no significant change in cell viability up to a treatment concentration of 300 μg/mL of the sample (B. vele).

### 1-2. Analysis of Fat Accumulation and TG Content

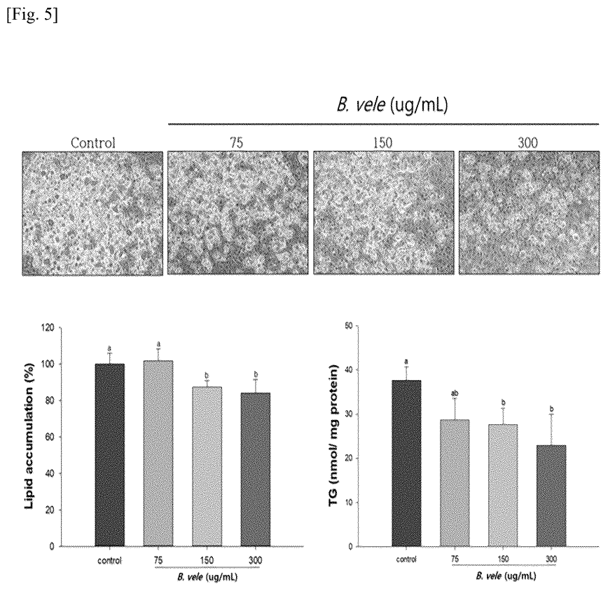

According to the Experimental Example 2-3, as a result of analyzing the effect of the sample on fat and TG accumulation, as shown in FIG. 5, the sample (B. vele) significantly inhibited fat and TG accumulation in a concentration-dependent manner, finding that the intracellular fat and TG accumulation was reduced by approximately 20% and 39%, respectively, in the 300 μg/mL treated group. In addition, from the results, it was revealed that the KMUO1 strain has an anti-obesity effect through inhibition of fat accumulation.

### 1-3. Analysis of Expression of Adipogenesis-Related Genes and FAS

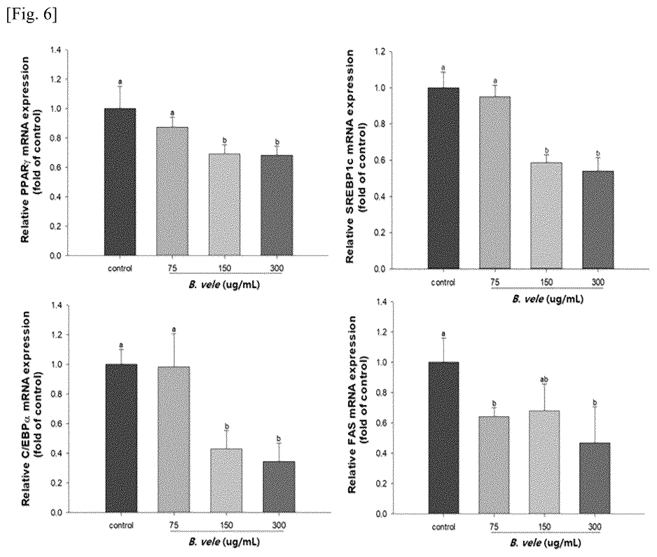

According to the Experimental Example 2-4, as a result of analyzing the effect of the sample on the expression of adipogenesis-related genes and FAS, as shown in FIG. 6, the sample (B. vele) inhibited the mRNA expression of PPARγ, C/EBPα, SREBP-1c, and FAS, and specifically, in the 300 μg/mL treated group, the mRNA expression was reduced by 32%, 65%, 46%, and 53%, respectively, compared to the control group.

### [Example 2] In Vivo Experiment

### 2-1. Analysis of the Body Weight and Food Efficiency

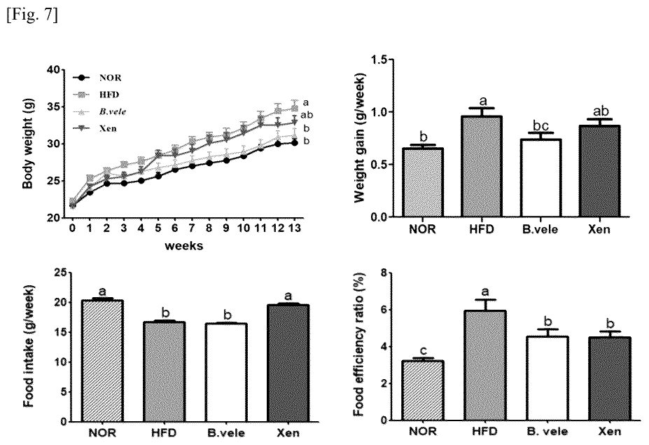

According to the Experimental Example 3-2, as a result of analyzing the effect of the sample on the body weight and food efficiency of the animal model, as shown in FIG. 7, it was found that the body weight of the high-fat diet group (HFD) significantly increased compared to the normal diet group (NOR), finding that obesity was induced. In addition, it was determined that the body weight significantly decreased in the sample administered group (B. vele) compared to the high-fat diet group (p<0.05). The weight gain was significantly higher in the high-fat diet group than in the normal diet group, showing that the sample administered group was significantly lower than the high-fat diet group (p<0.05). There was no difference in the food intake between the high-fat diet and sample administered groups, but the food efficiency was significantly low in the sample administered group (p<0.05). From the results, it was found that the sample had led to the reduction in digestion and absorption utilization rates of some nutrients or an increase in energy consumption.

### 2-2. Analysis of a Body Composition

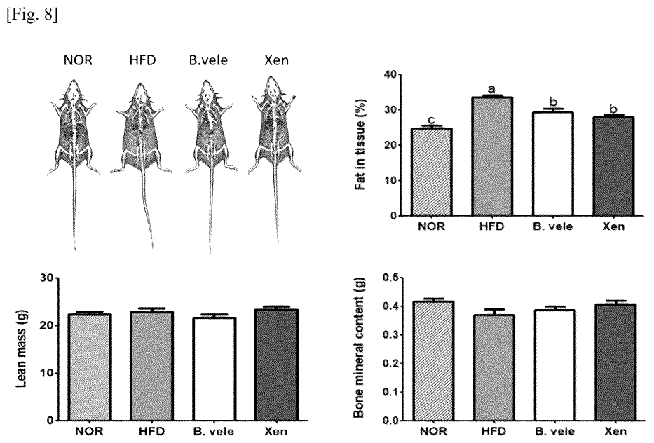

According to the Experimental Example 3-3, as a result of analyzing the effect of the sample on the body composition of the animal model, as shown in FIG. 8, it was found that body fat significantly increased in the high-fat diet group (HFD) compared to the normal diet group (NOR), and body fat significantly decreased in the sample administered group (B. vele) and positive control group (Xen). There were no significant differences in lean mass and bone mineral content between the experimental groups. From the results, it was found that the weight change by the sample was due to a decrease in body fat.

### 2-3. Analysis of Weights of Organs and Adipose Tissues

According to the Experimental Examples 3-4, as a result of analyzing the effects of the samples on organs and adipose tissues in the animal model, as shown in Table 1, there was no significant difference in the weights of the heart, liver, and spleen between the experimental groups. The kidney weight increased in the high-fat diet (HFD) group, but did not show statistical significance.

TABLE 1

| Weight (mg) | NOR | HFD | B. vele | Xen |
|---|---|---|---|---|
| Heart | 146 ± 24 | 150 ± 10 | 151 ± 23 | 157 ± 20 |
| Liver | 1104 ± 76 | 1085 ± 92 | 1047 ± 86 | 1121 ± 88 |
| Kidney | 341 ± 37 | 368 ± 46 | 355 ± 38 | 381 ± 46 |
| Spleen | 69 ± 12 | 69 ± 7 | 76 ± 13 | 73 ± 13 |

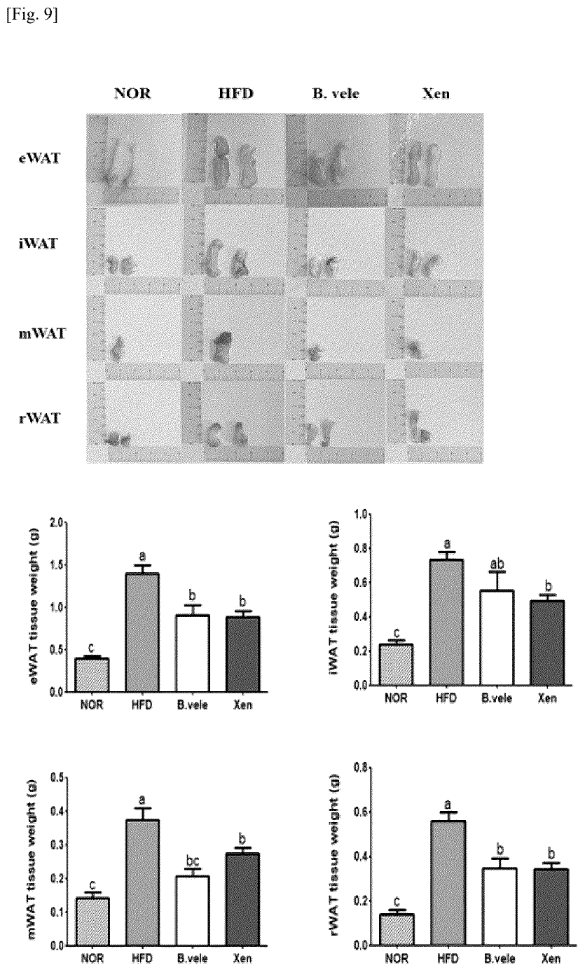

In addition, as shown in FIG. 9, the weight of all adipose tissues [inguinal white adipose tissue (iWAT), mesenteric white adipose tissue (mWAT), retroperitoneal white adipose tissue (rWAT), and epididymal white adipose tissue (eWAT)] significantly increased in the high-fat diet group (HFD) compared to the normal diet group (NOR), and the sample administered group (B. vele) showed a decrease in the weight of all adipose tissues compared to the high-fat diet group, showing a similar tendency to the positive control group (Xen) that had a decrease in the weight of all adipose tissues.

### 2-4. Blood Biochemical Analysis

According to the Experimental Examples 3-5, as a result of analyzing the effects of the sample on blood sugar, AST, ALT, BUN, and cholesterol of the animal model, as shown in Table 2, TG did not show a significant difference between the normal diet group (NOR) and the high-fat diet group (HFD), but TG in the positive control group (Xen) significantly decreased compared to the high-fat diet group (p<0.05). Total cholesterol (TCHO) and HDL-C significantly increased due to the high-fat diet, and LDL-C content significantly decreased in the sample administered group (B. vele) compared to the high-fat diet group. There were no significant differences between the experimental groups in respect to indicators for blood sugar, liver function abnormalities (AST and ALT), and renal toxicity (BUN).

TABLE 2

| Indicator | NOR | HFD | B. vele | Xen |
|---|---|---|---|---|
| TG (mg/dL) | 58.41 ± 16.43 | 59.61 ± 11.76 | 50.92 ± 26.35 | 30.56 ± 8.97 |
| Total cholesterol | 150.33 ± 21.91 | 178.80 ± 9.04 | 156.40 ± 10.11 | 173.60 ± 19.24 |
| (TCHO) (mg/dL) | | | | |
| HDL-C (mg/dL) | 113.74 ± 6.09 | 131.07 ± 6.70 | 133.96 ± 2.71 | 135.94 ± 2.71 |
| LDL-C (mg/dL) | 24.91 ± 18.01 | 35.81 ± 6.36 | 12.26 ± 9.28 | 31.55 ± 17.44 |
| Blood sugar | 209.29 ± 17.40 | 217.60 ± 66.05 | 191.60 ± 29.91 | 173.56 ± 21.07 |
| (mg/dL) | | | | |
| AST (U/L) | 66.43 ± 16.69 | 69.40 ± 11.72 | 59.00 ± 15.84 | 60.22 ± 17.38 |
| ALT (U/L) | 27.71 ± 3.99 | 26.00 ± 7.18 | 20.00 ± 9.19 | 20.00 ± 2.78 |
| BUN (mg/dL) | 31.81 ± 5.30 | 26.96 ± 4.90 | 27.96 ± 6.90 | 26.78 ± 4.00 |

All values are expressed as mean ± standard deviation.

### 2-5. Histological Analysis

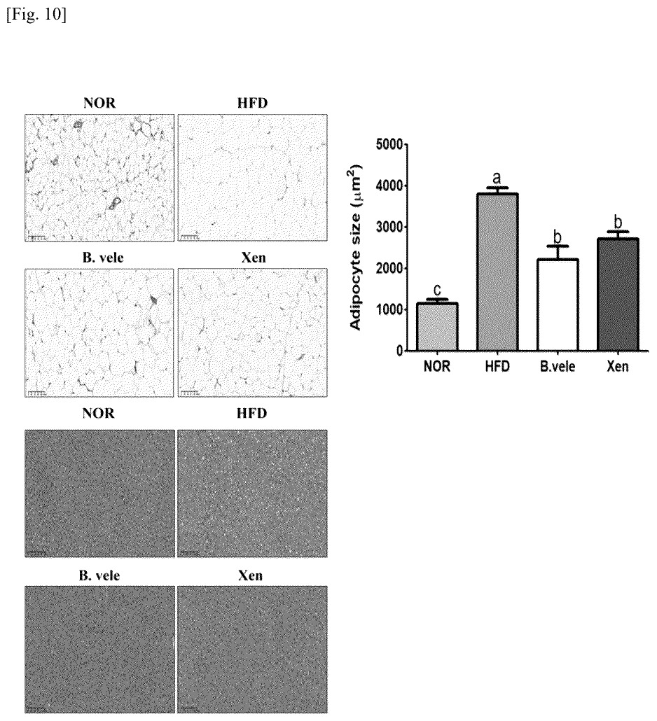

According to the Experimental Examples 3-6, as a result of analyzing the effect of the sample on the adipose tissue of the animal model, as shown in FIG. 10, in the epididymal white adipose tissue (eWAT), the size of the adipocytes in the high-fat diet group (HFD) significantly increased compared to the normal diet group (NOR), and in the sample administered group (B. vele) and the positive control group (Xen), the size of the adipocytes significantly decreased compared to the high-fat diet group, showing the inhibition of adipocyte hypertrophy. In addition, in the liver, the production of lipid droplets (white dots) increased in the high-fat diet group compared to the normal diet group, revealing that lipid droplets decreased in the sample administered group compared to the high-fat diet group.

### 2-6. Analysis of the Hepatic TG Content

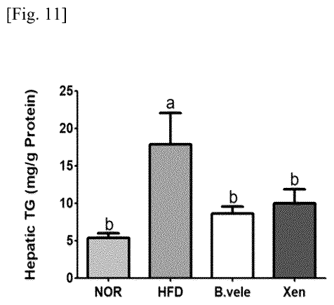

According to the Experimental Example 3-7, as a result of analyzing the effect of the sample on hepatic TG in the animal model, as shown in FIG. 11, the TG content in the high-fat diet group (HFD) significantly increased compared to the normal diet group (NOR), and the TG content in the sample administered group (B. vele) and the positive control group (Xen) significantly decreased compared to the high-fat diet group.

### 2-7. Analysis of Expression of Proteins Related to Hepatic Adipogenesis and Lipogenesis

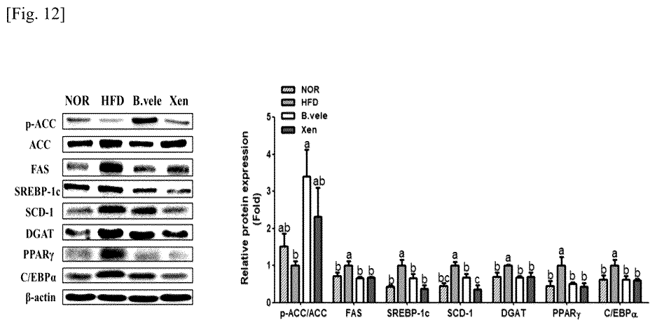

According to the Experimental Example 3-8, as a result of analyzing the effect of the sample on the expression of proteins related to hepatic adipogenesis and lipogenesis of the animal model, as shown in FIG. 12, in the sample administered group (B. vele), the expression of adipogenesis regulatory proteins (C/EBPα and PPARγ) was significantly reduced, and the expression of SREBP-1c, which regulates the expression of lipogenesis enzymes acetyl-CoA carboxylase (ACC) and fatty acid synthase (FAS), was shown to be reduced (P<0.05). Additionally, the expression of FAS, SCD-1, and DGAT, which are involved in the lipogenesis process, was significantly reduced (P<0.05). Phosphorylation of ACC, which inhibits ACC activation, was significantly increased, determining that the sample contributed to the inhibition of the lipogenesis process. The positive control group (Xen) also showed a similar tendency to the sample administered group (B. vele).

### 2-8. Analysis of Intestinal Microbes

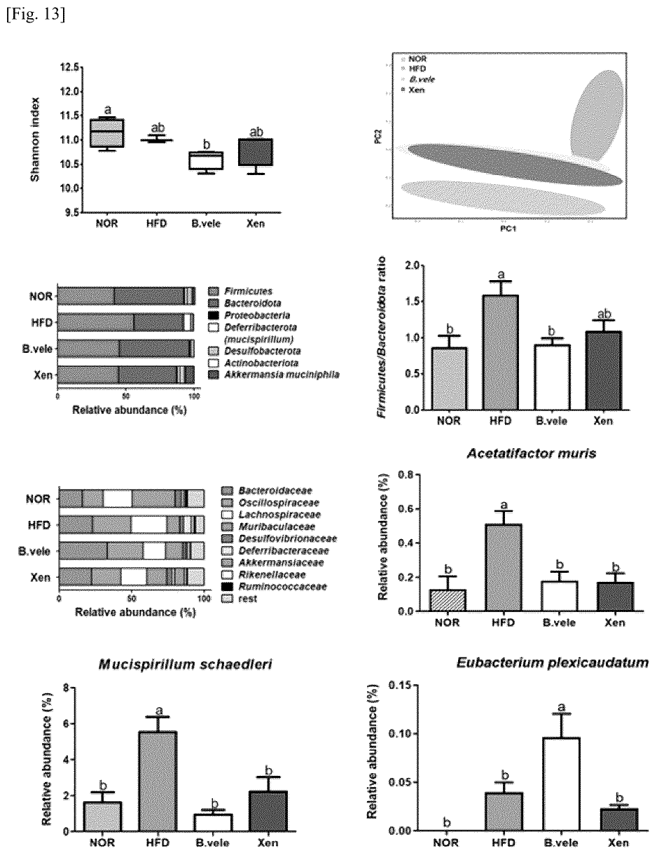

According to the Experimental Example 3-9, as a result of analyzing the effect of the sample on the intestinal microbes of the animal model, as shown in FIG. 13A, in the α-diversity analysis results that analyze the diversity of microorganisms present in a single sample, there was no significant difference observed in the Shannon index between the experimental groups. On the other hand, the β-diversity analysis results based on unweighted UniFrac principal coordinate analysis (PCA), which is frequently used in microbial colony analysis, showed significant differences between the experimental groups. Sample administration changed the composition of the intestinal microbes to be similar to that of the normal diet group (NOR).

Additionally, the relative abundance of Deferribacterota significantly increased in the high-fat diet group (HFD) and significantly decreased in the sample administered group and positive control group (Xen). In structural microbial colony analysis at the phylum level, the relative abundance of Firmicutes increased due to a high-fat diet but that of Bacteroidota decreased, showing that the Firmicutes/Bacteroidota (F/B) ratio increased, while the F/B ratio decreased in the sample administered group. At the family level, the relative abundance of Muribaculaceae in the high-fat diet group significantly decreased compared to the normal diet group. In the sample administered group, the relative proportion of Lachnospiraceae decreased, whereas that of Muribaculaceae increased. At the species level, the relative abundance of Acetatifactor muris and Mucispirillum schaedleri significantly increased in the high-fat diet group compared to the normal diet group. In the sample administered group, the relative abundance of Acetatifactor muris and Mucispirillum schaedleri significantly decreased compared to the high-fat diet group, whereas that of Eubacterium plexicaudatum significantly increased.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

1. A pharmaceutical composition for preventing or treating obesity, comprising a fermentation culture supernatant of a Bacillus velezensis strain, a concentrate thereof, a dried product thereof, a fermentation metabolite thereof, or a mixture thereof as an active ingredient.

2. The pharmaceutical composition of claim 1, wherein the strain is a Bacillus velezensis KMU01 strain deposited under deposit number KCTC11751BP.

3. The pharmaceutical composition of claim 1, wherein the pharmaceutical composition further comprises killed cells or spores of Bacillus velezensis.

4. The pharmaceutical composition of claim 1, wherein the fermentation metabolite is short chain fatty acids (SCFAs), organic acids, or amino acids.

5. The pharmaceutical composition of claim 4, wherein the short-chain fatty acid is butyric acid or propionic acid.

6. The pharmaceutical composition of claim 4, wherein the amino acid is phenylalanine, which is an aromatic amino acid, or valine, which is a branched amino acid.

7. The pharmaceutical composition of claim 1, wherein the pharmaceutical composition regulates one or more intestinal microbes selected from the group consisting of Acetatifactor muris, Mucispirillum schaedleri, and Eubacterium plexicaudatum.

8. The pharmaceutical composition of claim 1, wherein the obesity is one or more selected from the group consisting of visceral obesity, abdominal obesity, systemic obesity, and partial obesity.

9. A health functional food composition for preventing or ameliorating obesity, comprising a fermentation culture supernatant of a Bacillus velezensis strain, a concentrate thereof, a dried product thereof, a fermentation metabolite thereof, or a mixture thereof as an active ingredient.

10. A food composition for preventing or ameliorating obesity, comprising a fermentation culture supernatant of a Bacillus velezensis strain, a concentrate thereof, a dried product thereof, a fermentation metabolite thereof, or a mixture thereof as an active ingredient.

11. A health functional food composition for reducing body fat or blood cholesterol, comprising a fermentation culture supernatant of a Bacillus velezensis strain, a concentrate thereof, a dried product thereof, a fermentation metabolite thereof, or a mixture thereof as an active ingredient.

12. A method of preventing or treating obesity, comprising treating a subject with the pharmaceutical composition according to claim 1.

13. The method of claim 12, wherein the method of preventing or treating obesity derives an effect of ameliorating obesity or reducing visceral fat by decreasing a relative abundance of Acetatifactor muris or Mucispirillum schaedleri strains in the intestine and increasing a relative abundance of a Eubacterium plexicaudatum strain in the intestine.
